(19) United States
(12) Patent Application Publication
(10) Pub. No.: US 2010/0297688 A1
(43) Pub. Date: Nov. 25, 2010

(54) METHOD FOR DIAGNOSING OR PREDICTING SHORT STATURE IN HUMANS

(76) Inventor: Svetlana TEN, Staten Island, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 12/783,944

(22) Filed: May 20, 2010

Related U.S. Application Data
(60) Provisional application No. 61/180,255, filed on May 21, 2009.

(51) Int. Cl. C12Q 1/02

(52) U.S. Cl. 435/29

(57) ABSTRACT

An improved assay for measuring the release of endogenous growth hormone (GH) is described. The method focuses on determining the presence of an immediate release pool (IRP) of GH, and the extent to which it is rapidly discharged into the circulation of humans, including children. A larger and less labile pool responds continuously to long term stimulation. The method considers that determining the ten minute AUC measurement, i.e., ten minutes after induction of GH release by administering growth hormone releasing hormone (GHRH) will reveal an immediate release pool of GH. As further disclosed herein, the IRP of GH has a higher correlation with peak GH release than the conventional 120 minute time point. Further the AUC₁₀ correlates with height standard deviation measurements whereas the AUC₁₂₀ does not.

### CROSS-REFERENCE TO RELATED APPLICATIONS

This application claims benefit of U.S. Provisional Application No. 61/180,255 filed on May 21, 2009.

### FIELD OF THE INVENTION

The invention relates to the fields of endocrinology and physiology as means for predicting a predisposition to, or diagnosing of, a medical condition directly or indirectly associated with the release and/or action of growth hormone (GH).

### BACKGROUND OF THE INVENTION

Growth hormone (GH) is a peptide hormone that stimulates growth and cell reproduction in humans and other animals. GH is a single chain polypeptide hormone which is synthesized, stored, and secreted by the somatotroph cells located within the anterior pituitary gland; endogenous GH that is produced natively in animals, including humans and other mammals, is sometimes referred to as somatotrophin. GH, especially human GH can be prepared by recombinant DNA technology, and is abbreviated rhGH (recombinant human growth hormone). The major endogenous form of human GH is a polypeptide of 191 amino acids in length, which has a molecular weight of 22,124 daltons. The structure includes four helices necessary for functional interaction with the GH receptor.

Peptide factors released by neurosecretory nuclei of the hypothalamus regulate the release of GH from the anterior pituitary. One factor, GHRH (i.e., Growth hormone releasing hormone) is a potent stimulator of GH secretion, while another peptide SS (somatostatin) inhibits the release of GH into the portal venous blood surrounding the pituitary. Although the balance between GHRH and somatostatin is the major regulator of GH secretion by somatotrophs, this balance itself may be affected by other physiological stimulators (e.g exercise, nutrition, sleep) and inhibitors of GH secretion (e.g. free fatty acids). Effects of growth hormone on the tissues of the body can generally be described as anabolic or growth-stimulating resulting in increases in tissue mass rather than using up tissue by various degradative processes, thereby diminishing cell and tissue mass in the body.

A large increase in bone mass occurs during childhood and puberty via endochondral bone formation; the replacement of cartilage by bone tissue. This is the major process by which human long bones, e.g., the bones making up the extremities, reach their adult length. A gradual increase in bone mass is then seen until peak bone mass is reached at 20-30 yrs of age. Subsequently, bone mass decreases with an accelerated bone loss seen in females after menopause. Bone remodeling is regulated by a balance between bone resorption and bone formation, the latter process being one in which GH is known to play a role. A net gain of skeletal mass due to new bone formation caused by GH was first shown in adult dogs. After treatment with GH for 3 months a 2% increase in cortical bone mass, as assessed by histomorphometry, was found.

Stimulating the increase in height in childhood is the most widely known effect of GH, and appears to be stimulated by at least two mechanisms. GH directly stimulates division and multiplication of chondrocytes of cartilage. These are the primary cells in the growing ends (epiphyses) of children's long bones (arms, legs, digits). However, GH also indirectly stimulates bone growth via the production of insulin-like growth factor 1 (i.e., IGF-1) a hormone homologous to proinsulin. The indirect IGF-1 regulated effects of GH are mediated by the liver, which is the principal site of IGF-1 production. IGF-1 has growth-stimulating effects on a wide variety of tissues, and in some cases, the target tissue itself is an additional source of IGF-1, making it apparently both an endocrine and an autocrine/paracrine hormone. Serum GH concentrations fluctuate thereby rendering random sampling of serum GH an inconclusive test of dysfunction of the GH/IGF-1 axis. Tests for stimulating GH secretion have been established to assess the maximum serum GH concentration that can be released in response to an exogenous stimulus. Various pharmacological agents can induce GH release, among the most commonly used are are glucagon, clonidine, levodopa, arginine, insulin-induced hypoglycemia and growth hormone releasing hormone (GHRH). These GH provocative tests, or GH stimulating tests (i.e., GHST), are problematic in children, however, as they do not reliably identify those with hypothalamic dysfunction. Although GH provocative tests were introduced in clinical practice more than 40 years ago, consensus on the use of these tests has not been achieved regarding time intervals to employ a test, which test to use, and the predictive value for a clinical response. Studies have shown that provocative GH testing frequently does not correlate with endogenous GH secretion (see e.g., Bercu, B. B., et al., (1986) J. Clinical. Endocrinol Metab. 63, 709-716; Bell, J. J. et al., (1998) Pediatrics 102(2) 518-520). One reason for the lack of correlation may be that there are no standardized GH testing methods used by endocrinologists. For example, the drug or hormone used to stimulate GH release can vary, e.g., glucagon, clonidine, levodopa, arginine, insulin-induced hypoglycemia and growth hormone releasing hormone (GHRH), in addition to the time frame employed for collecting blood for GH measurement. It is common for endocrinologists to collect blood samples from about 30 minutes post-stimulation, until from about 90 minutes to about 180 minutes post-stimulation (e.g., see Spiliotis, B. E., et al., (2008) Horm Res 70:215-223 Mahajan, T., et al., (2000) J. Clin Endocrinol Metab. 85:1473-1476).

The prior GH stimulation tests were established to assess the peak secretory levels of GH by the anterior pituitary in response to a pharmacological stimulus. A review of the relevant literature reveals significant problems with routinely used GH post-stimulation time-course measurements relying on GH determinations taken as long as 2 hour after pharmacologically provoking GH release into the blood. The current status of provocative GH testing is acknowledged to have its downside; e.g., it is an invasive testing procedure in that numerous blood samples are taken from a subject during the course of administering the test, thereby providing discomfort and additional potential risks. Further, in some instances the results have been reported to not be reproducible even when testing in the same patient, and often there is often conflicting results with other stimulation test results or growth data (Hardin DS, et al., (2007) Clin Endocrinol, 66: 85-94) which underscore the unreliability of provocative GH testing. To some degree, this may be due to the arbitrary assignment of 10 ng/ml as the “normal” vs “subnormal” levels of serum GH. Further, in most sites of administering a provocative GH test, it is likely to be expensive and labor-intensive (see Tauber M., et al., (1997) J. Clin Endocrinol Metab 82: 352-356). These issues are apparently reflected in the declining use of GH stimulation tests over the past two decades, from a high of 89% among endocrinologists between 1987 and 1989, to about 52% in 2002. A recent survey of practices prescribed among U.S. members of the Lawson Wilson Pediatric Endocrinology Society conclude that the GH stimulation test was not the best way to determine if a pre-adult, e.g., a child or teen would benefit from GH therapy (Wilson DM, et al., (2005) Growth Horm IGF Res. 15(Suppl A): p. S21-25).

An additional factor may be complicating the interpretation of GH stimulation test results. It now appears that there are two pools of GH. Kinetic analysis of rat GH synthesis and secretion suggests that intracellular rat GH may exist as two distinct pools that are functionally segregated into at least two kinetically distinguishable compartments (Fukata, et al., (1985) Endocrinology 117: 457:467). One compartment houses an immediate release pool (IRP) that responds quickly to secretory stimuli and can be quickly exhausted. A larger and less labile pool (i.e., more slowly turning over) responds continuously to long term stimulation. However, in view of the early phase of GH secretion, it seemed that selecting early time points for collecting blood for GH assay, i.e., between 0-15 minutes after stimulating GH secretion would not be useful. An invention that appreciates that early phase secretion for GH assessed at 0 to 15 minutes would help kinetically to distinguish the two intracellular pools of GH. In addition, if an IRP-like pool of GH were observed in humans and other primates, it could provide the basis for a more reliable indicator of the endogenous GH available for rapid release at any given time. The IRP-like pool could also provide an improved indicator or predictor of GH deficiency and short stature in adulthood. Further, the fact that the blood samples would be taken over a shorter period of time than in conventional assays may translate into a less stressful experience for the patient.

### SUMMARY OF THE INVENTION

In accordance with the needs described above in the area of endocrinology, and specifically pediatric endocrinology, it is one object of the invention to provide an improved assay or test for determining the status of GH secretion by a subject's or patient's anterior pituitary.

Within the context of accurately measuring GH release, it is an object of the invention to collect patient samples, e.g., blood, urine and the like, from between 1 minute and 20 minutes of inducing GH secretion. The collecting of the patient sample is preferably between 5 and 15 minutes after inducing GH secretion, and most preferably from at about 9 minutes, 10 minutes or 11 minutes. It is emphasized that patient samples will likely be taken at additional times after GH secretion is induced for reasons including the fact that early GH release may occur somewhat later in some patients.

In the context of accurately determining the GH release kinetics of a pre-adult or young adult, it is an object of the invention to diagnose GH deficient patients that would benefit from GH therapy, i.e., the administration of exogenous forms of GH. It is an additional object of the invention to provide a rapid method to identify candidates that may respond to GH therapy. A further object of the invention is to develop a standardized test wherein physicians, nurses or other healthcare workers can help identify patients with a predisposition to maintaining a short stature in adulthood as evidenced by a deficiency in GH secretion into the blood.

It is described herein that the aforementioned goals and objects of the invention can be met by a method for predicting whether a pre-adult human will likely retain a short stature as an adult due to GH deficiency, by administering to a pre-adult human a GH secretion-inducing compound in an amount sufficient to induce GH secretion; collecting one or more blood samples from the pre-adult human at between 0 and about 10 minutes after administering of the GH secretion-inducing compound; generating a calculated area under the curve (AUC) value for the GH released into the blood at about 10 minutes (AUC₁₀) after administering the GH secretion-inducing compound, and comparing the calculated AUC₁₀ value to corresponding AUC₁₀ values from known normal and known GH-deficient individuals, and wherein the calculated AUC₁₀ value, being encompassed by a range of corresponding AUC₁₀ values from GH-deficient individuals, is an indicator that the pre-adult human is likely to retain a short stature.

### BRIEF DESCRIPTION OF THE DRAWINGS

FIG. 1 provides, in tabular form, the GH release data at the indicated times after inducing GH release. The columns with AUC indicate the accumulation of secreted GH up to that time point. Area Under the Curve is a statistical means of summarizing information from a series of measurements on one individual. It represents the total uptake of whatever has been administered. Areas under the curve (AUC) for GH were calculated according trapezoid rule (Tai MM 1994 A mathematical model for the determination of total area under glucose tolerance and other metabolic curves. Diabetes Care 17:p 152-4).

FIG. 2(a)-(d) illustrates the correlation between (a) IGF-1 and AUC₁₀, (b) IGF-1 and AUC₁₂₀, (c) AUC₁₀ A and HT SDS and (d) AUC₁₀ and the GH Peak.

### DETAILED DESCRIPTION OF THE INVENTION

In the context of the method of the present invention, the term “pre-adult” generally refers to any individual patient or subject that is still at an age where detectable skeletal growth can be observed. In one embodiment, the pre-adult is a newborn child. In another embodiment a pre-adult is contemplated to include individuals from birth to about 18 years of age. The term “GH deficient” refers to individuals who have blood levels of any active form of endogenous GH that persons of ordinary skill in the endocrinological and medical arts would consider or readily understand to be abnormally low, thereby resulting in medically recognized conditions such as growth hormone deficiency due to genetic mutations, trauma or cranial tumors. Accordingly, a biochemical definition of a GH-deficient individual is considered to have peak of blood GH level below 10 μg/ml during GH stimulation test in male and female pre-adults. In contrast, normal pre-adults generally have peak GH levels higher than 10 μg/ml in male and female pre-adults. In the context of the description of the process of the invention, a normal pre-adult will not possess GH-dependent pathologies, including short stature (Wilson DM, F. J., A brief review of the use and utility of growth hormone stimulation testing in the NCGS: Do we need to do provocative GH testing?. Growth Horm IGF Res, 2005. 15(Suppl A): p. S21-25.).

Inasmuch as a GHRH test is the most physiological test presently available, the GHRH test of the present invention will be capable of segregating normal subjects from GH deficient patients. The inventor recognizes that as GHRH induces GH peaks consistently in the first 30 min, a 10 min area under the curve of GH secretion can be used for earlier diagnosis at least as reliably as the peak levels during 120 minute test. Thus, the invention surprisingly and dramatically reduces the time it takes to segregate normal subjects from GH deficient patients by about 12 times. The invention thus facilitates a rapid, reliable cost-effective determination of GH-deficient individuals, that requires minutes and not hours to complete, thus saving patients the agony of multiple blood samplings and wasted time. In addition, the experimental evidence indicates that the earlier time points, e.g., 10 min blood samples, has a higher correlation, indicating that it is more accurate than conventional methods of diagnosing GH-deficiency.

Patients. A GHRH test was conducted after overnight fasting, modified with samples that were obtained at 3, 5, and 10 minutes in addition to routine protocol collection of samples at 30 minutes, 60 minutes, 90 minutes and 120 minutes following GHRH injection (GHRH1-29: GEREF, SERONO, Italy; 1 μg/kg IV at 0 min). A total of 19 children (8.6±3.2, range from 2.5 yrs to 14 yrs, 5 girls) who suffered SS (short stature) (Ht−2.5±0.6 SDS) were administered GHRH 1 μg/kg IV at 0 min and blood samples were obtained at 0, 3, 5, 10, 30, 60, 90 and 120 min. GH was determined in all samples.

Blood sample processing. GH and IGF-1 were analyzed at Esoterix (Texas, U.S.). IGF1 was measured by radioimmunoassay after acid-ethanol extraction and with IGF2 blocking. A 100 uL of sample was initially used for the procedure. Growth hormone was tested by ICMA, requires min amount of 0.2 ml of serum, preparation include 1 mL serum, separate within one hour that was shipped frozen in plastic vial (http://www.esoterix.com/prodserv/test_menu!)

AUC measurements for GH were calculated according trapezoid rule (Tai MM 1994 A mathematical model for the determination of total area under glucose tolerance and other metabolic curves. Diabetes Care 17:p 152-4).

The correlation between the AUC at a specific time point and the correlation with GH level, IGF-1 level or HT SDS was performed by regression analysis.

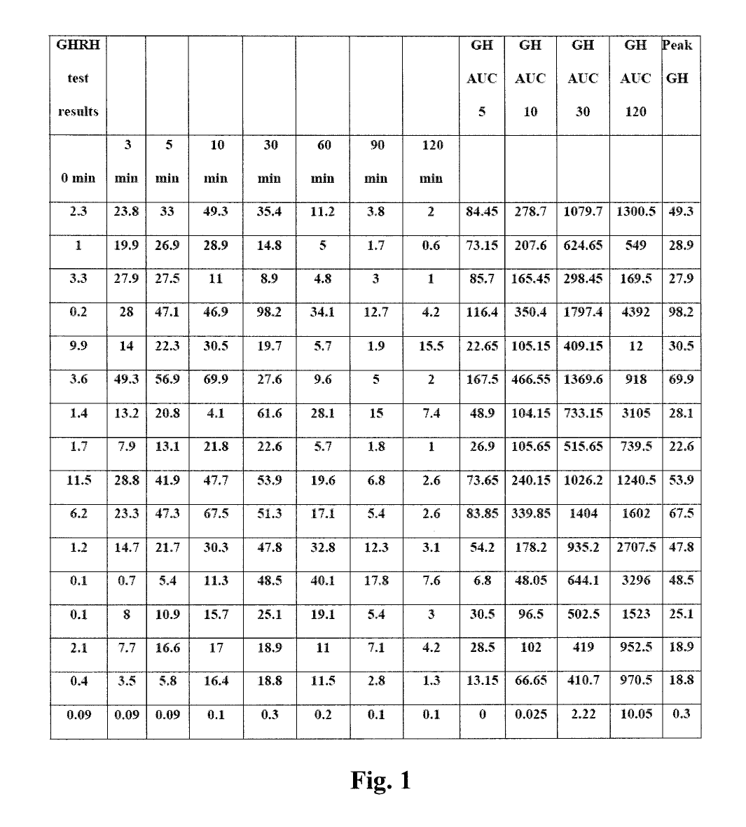

FIG. 1 illustrates the amount of released endogenous GH levels in blood samples taken at the indicated times after injecting the subject with GHRH. The GH values in the columns representing specific time points in minutes, 0, 3, 5, 10, 30, 60, 90 and 120, when blood samples were taken are expressed as ng/ml, as were the peak GH measurements shown in the last column.

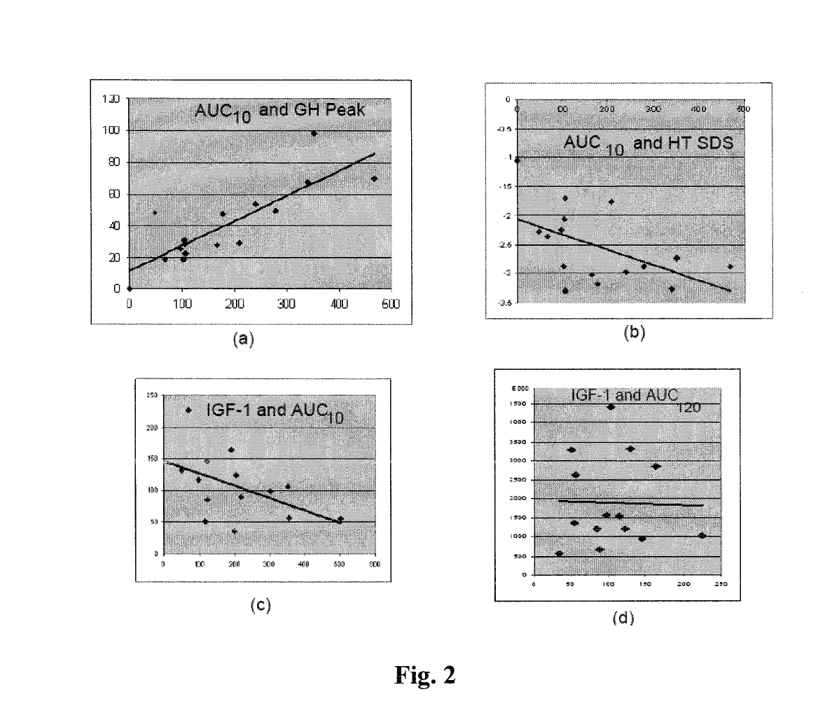

The AUC values were calculated at the corresponding time points and are provided in the table in arbitrary units representing the integrated area under the curve at the indicated time point. The results establish that by measuring blood or serum GH levels at early time points, e.g., at 3, 5 and 10 minutes after administering GHRH to a subject, rather than the currently used methodology used in the art it was possible to demonstrate an unexpected and surprisingly significant increase in the accuracy of identifying subjects presenting a short stature. As shown in FIG. 2a, the AUC₁₀ showed a very strong correlation with peak GH release (r=0.83, p<0.001). In contrast, the conventionally used scheme, represented by the 120 minute time point showed a significantly less pronounced correlation with peak GH release, (r=0.62, p=0.01) using the AUC₁₂₀ value, rather than AUC₁₀.

Therefore, measuring one or more blood or serum samples for GH released within about 10 minutes post-stimulation with GHRH provides a superior indication of releasable GH present in an immediate release pool than measurements taken at later times.

FIG. 2b illustrates a surprising and unexpected result that the AUC₁₀ actually correlated with HT SDS (r=−0.52, p=0.038), and, just as surprising is that the values calculated for AUC₁₂₀ did not correlate with HT SDS at all. AUC₁₀ correlated with IGF-1 baseline (FIG. 2c) before treatment, while AUC₁₂₀ did not correlate with IGF-1 levels (FIG. 2d). In sum, the GH measurements taken at times up to about 10 minutes, i.e., AUC₁₀ post-stimulation with GHRH are far more correlative in pre-adults with short stature than values taken up to 120 minutes, i.e., AUC₁₂₀ after GHRH stimulation.

The GH stimulation tests were established to assess the peak secretory levels of GH by the anterior pituitary in response to a pharmacological stimulus. A current review of literature shows significant problems with routinely used GH post-stimulation time-course measurements that rely on GH determinations taken as long as 2 hour after provoking GH release into the blood. The current status of provocative GH testing is acknowledged to have its downside; e.g., it is an invasive testing procedure in that numerous blood samples are taken from a subject during the course of administering the test, thereby providing discomfort and additional potential risks. Further, in some instances the results have been reported to not be reproducible even when testing in the same patient, and often there is often conflicting results with other stimulation test results or growth data (Hardin D S, et al., (2007) Clin Endocrinol, 66: 85-94) which underscore the unreliability of provocative GH testing. To some degree, this may be due to the arbitrary assignment of 10 ng/ml as the “normal” vs “subnormal” levels of serum GH. Further, in most sites of administering a provocative GH test, it is likely to be expensive and labor-intensive (see Tauber M., et al., (1997) J. Clin Endocrinol Metab 82: 352-356). These issues are apparently reflected in the declining use of GH stimulation test over the past two decades, from a high of 89% among endocrinologists between 1987 and 1989, to about 52% in 2002. A recent survey of practices prescribed among U.S. members of the Lawson Wilson Pediatric Endocrinology Society conclude that the GH stimulation test was not the best way to determine if a pre-adult, e.g., a child or teen would benefit from GH therapy (Wilson D M, et al., (2005) Growth Horm IGF Res. 15(Suppl A): p. S21-25).

Research over the last decade or so has altered our understanding of the GH-IGF-1 axis. Analysis of the data obtained from GH registries has furthered our understanding of what variables to consider in determining candidates that are likely to experience a meaningful GH therapeutic response. Rosenfeld R, (1995) J Clin Endocrinol Metab 80:1532-1540; National Cooperative Growth Study (NCGS) of Optimal Nutropin AQ and Nutropin Dosing in Pubertal Growth Hormone-Deficient (GHD) Patients (2006)). These studies, and others demonstrate that GH therapy can be successful, but not in all cases. Thus, alternative approaches have been suggested, such as titrating the GH dose to serum IGF-1 levels (Cohen P, (2007) J Clin Endocrinol Metab 92:2480-2486)). The basis of this approach has had some theoretical appeal because circulating IGF-1 levels are regulated by GH and tend to be low in GH deficiency and in GH insensitivity and high in states of GH excess. In children treated with GH, IGF-1 levels rise in a dose-dependent manner (1). In addition, circulating IGF-1 mediates many of the physiological effects of GH. Other studies have revealed that even non-GH-deficient short stature can be responsive to GH treatment, e.g., in Prader-Willi syndrome (Corrias, A, et al., (2000) J Endocrinol Invest. February; 23(2):84-9 2000), Turner syndrome (Saenger P, (1996) J Clin Endocrinol Metab 84(12) 4345-4348) and Noonan syndrome (Raaijmakers, R, et al., J Pediatr Endocrinol Metab. (2008) 21(3):267-73).

In sum, it appeared that a re-examination of the provocative GH secretion test was warranted. Analysis of the literature has shown that among the many variants of the GH stimulation tests used, we did not identify a single clinical application based on the measurements of an immediate release pool of stored GH. As described above, such approach of GH stimulation test was unreliable and was not accurately predictive of growth rate, GH resistance, IGF-1 levels or any change in HT SDS upon administering GH treatment, especially in highly prevalent group of idiopathic short stature (ISS).

AUC₁₀ correlated with HT SDS (r=−0.52, p=0.03), while AUC₁₂₀ did not correlate with HT SDS in a statistically significant way. With respect to previous studies by others, the correlation was stronger than reported in previous studies. Rakover et al. evaluated 164 prepubertal children with short stature with GHST, and the HT SDS correlated negatively with basal GH values in all subjects (r=−0.358, P<0.0001), in normal responders (r=−0.45, P<0.0001) and in exaggerated responders (r=−0.341, P<0.0001), but not in the GH deficient group (Rakover Y, et al., (1999) Eur J Endocrinol (2002) 146: 319-323). Bright and colleagues (Bright GM, (1999) Pediatrics, 104(4 Pt 2): p. 1028-1031) retrospectively examined the relationship between GH peak response to stimulation testing and the gain in HT SDS in 236 prepubertal children with IGHD or ISS. They demonstrated that there was essentially no relationship between the peak GH response in stimulation testing and the change in HT SDS in response to GH treatment in patients with peak GH responses greater than 2-3 ng/ml.

As clearly supported by the experimental results contained herein, the method of the present invention evaluates the acute GH response in the first 0 to about 15 min after GHRH administration, as this method is far more predictive than methods dependent upon later time points, up to and including the 120 min response. Correlation of the AUC values with IGF-1 and HT SDS at baseline further support the conclusion that this approach to testing is more physiological, reliable, and easier for the pre-adult patient. Accordingly, in one embodiment a diagnostic test for determining whether or not a pre-adult will have short stature is provided. The diagnostic method employs a GH secretion stimulator, blood sample capture at about 10 minutes, AUC₁₀ calculation and a comparison with established AUC₁₀ values in normal and GH-deficient individuals, to determine or predict short stature in adulthood from an earlier vantage point than ever contemplated before.

### REFERENCES

1. Castro A I, L. M., Peino R, Kelestimur F, Dieguez C, Casanueva F F, A single growth hormone determination 30 minutes after the administration of the GHRH plus GHRP-6 test is sufficient for the diagnosis of somatotrope dysfunction in patients who have suffered traumatic brain injury. J Endocrinol Invest, 2007. 30(3): p. 224-229.
2. Wilson D M, F. J., A brief review of the use and utility of growth hormone stimulation testing in the NCGS: do we need to do provocative GH testing?. Growth Horm IGF Res, 2005. 15(Suppl A): p. S21-25.
3. Hardin D S, W. J., Butsch R, Huett B, Current prescribing practices and opinions about growth hormone therapy: results of a nationwide survey of paediatric endocrinologists. Clin Endocrinol, 2007. 66: p. 85-94.
4. Bright G M, J. J., Lima J, Blethen S L, Growth hormone stimulation test results as predictors of recombinant human growth hormone treatment outcomes: preliminary analysis of the national cooperative growth study database. Pediatrics, 1999. 104(4 Pt 2): p. 1028-1031.
5. Ranke M B, L. A., Price D A, Darendeliler F, Albertsson-Wikland K, Wilton P, Reiter E O; KIGS International Board, Age at growth hormone therapy start and first-year responsiveness to growth hormone are major determinants of height outcome in idiopathic short stature. Horm Res, 2007. 68(2): p. 53-62.
6. Marin G, D. H., Barnes K M, Blackwell B J, Cassorla F G, Cutler G B Jr, The effects of estrogen priming and puberty on the growth hormone response to standardized treadmill exercise and arginine-insulin in normal girls and boys. J Clin Endocrinol Metab 1994. 79: p. 537-541.
7. Chemaitilly W, T. C., Souberbielle J C, Brauner R, Assessing short-statured children for growth hormone deficiency. Horm Res, 2003. 60: p. 34-42.
8. Tauber M, M. P., Pienkowski C, Jouret B, Rochiccioli P, Growth hormone (GH) retesting and auxological data in 131 GH-deficient patients after completion of treatment. J Clin Endocrinol Metab, 1997. 82(2): p. 352-356.
9. Rakover Y, S. A., Lavi I, Masalha R, Shlomo I B, Can exaggerated response to a GH provocative test identify patients with partial GH insensitivity syndrome? Eur J Endocrinol, 2002. 146: p. 319-323.
10. Prakash A, G. K., Sermorelin: a review of its use in the diagnosis and treatment of children with idiopathic growth hormone deficiency. BioDrugs 1999. 12(3): p. 139-157.
11. Martinelli C E, S. M. S., Previato J K, Figueira M, Rangel Montenegro A P, Miraki-Moud F, Betancourth S, Moreira A C, Savage M O, Camacho-Hübner C, Final height in patients with idiopathic short stature and high growth hormone responses to stimulation tests. Horm Res, 2007. 67(5): p. 224-230.

What is claimed is:

1. A method for identifying growth hormone (GH) deficiency in a pre-adult human, comprising:
administering to a pre-adult human at least one GH secretion-inducing compound in an amount sufficient to induce GH secretion;
collecting one or more blood samples from the pre-adult human at between 0 and about 10 minutes after administering the at least one GH secretion-inducing compound;
generating a calculated area under the curve (AUC) value for the GH released into the blood at about 10 minutes (AUC₁₀) after administering the at least one GH secretion-inducing compound, and
comparing the calculated AUC₁₀ value to corresponding AUC₁₀ values from normal and GH-deficient individuals, and wherein the calculated AUC₁₀ value being encompassed by a range of corresponding AUC₁₀ values from GH-deficient individuals, is an indicator of GH deficiency.

2. A method for determining whether a pre-adult human will likely retain a short stature as an adult due to GH deficiency, comprising:
administering to a pre-adult human at least one GH secretion-inducing compound in an amount sufficient to induce GH secretion;
collecting one or more blood samples from the pre-adult human at between 0 and about 10 minutes after administering the at least one GH secretion-inducing compound;
generating a calculated area under the curve (AUC) value for the GH released into the blood at about 10 minutes (AUC₁₀) after administering the at least one GH secretion-inducing compound, and
comparing the calculated AUC₁₀ value to corresponding AUC₁₀ values from normal and GH-deficient individuals, and wherein the calculated AUC₁₀ value being encompassed by a range of corresponding AUC₁₀ values from GH-deficient individuals, is an indicator that the pre-adult human is likely to retain a short stature.

3. A method for determining whether a pre-adult human is likely respond to growth hormone (GH) therapy, comprising:
administering to a pre-adult human at least one GH secretion-inducing compound in an amount sufficient to induce GH secretion;
collecting one or more blood samples from the pre-adult human at between 0 and about 10 minutes after administering the at least one GH secretion-inducing compound;
generating a calculated area under the curve (AUC) value for the GH released into the blood at about 10 minutes (AUC₁₀) after administering the at least one GH secretion-inducing compound, and
comparing the calculated AUC₁₀ value to corresponding AUC₁₀ values from normal and GH-deficient individuals, and wherein the calculated AUC₁₀ value being encompassed by a range of corresponding AUC₁₀ values from GH-deficient individuals, is an indicator that the pre-adult human will respond to GH therapy.

4. The method of claim 3 wherein the at least one GH secretion-inducing compound is selected from the group consisting of growth hormone releasing hormone (GHRH), GHRH1-29, arginine, L-DOPA, ghrelin, growth hormone releasing hexapeptide (GHRP-6), prostaglandin E1, N⁶, 2′-O-dibutyryl-cAMP (DBcAMP), N-(2,6-dichlorophenyl)-4,5-dihydro-1H-imidazol-2-amine (clonidine), 5-hydroxy-tryptamine (5-HT), and β-endorphin.

5. The method of claim 4, wherein the at least one GH secretion-inducing compound is GHRH1-29.

6. The method of claim 5, wherein the GHRH1-29 is administered intravenously.

7. The method of claim 3, wherein blood samples are taken at about 0 min and about 10 min.

8. The method of claim 7, further comprising at least one additional time point between about 0 and about 10 min.
